(19) United States
(12) Patent Application Publication
(10) Pub. No.: US 2024/0158416 A1
(43) Pub. Date: May 16, 2024

(54) FUSED DIHYDRO-4H-PYRAZOLO[5,1-C][1,4]OXAZINYL COMPOUNDS AND ANALOGS FOR TREATING CNS DISORDERS

(71) Applicant: Sumitomo Pharma America, Inc., Marlborough, MA (US)

(72) Inventors: Milan CHYTIL, Acton, MA (US); Michele HEFFERNAN, Holliston, MA (US); Linghong XIE, Southborough, MA (US)

(73) Assignee: Sumitomo Pharma America, Inc., Marlborough, MA (US)

(21) Appl. No.: 18/554,066

(22) PCT Filed: Apr. 6, 2022
(86) PCT No.: PCT/US2022/071564

§ 371 Date: Oct. 5, 2023

Related U.S. Application Data
(60) Provisional application No. 63/172,137, filed on Apr. 8, 2021.

(51) Int. Cl. C07D 498/14; C07D 498/04; C07D 513/14

(52) U.S. Cl. CPC C07D 498/14; C07D 498/04; C07D 513/14

(57) ABSTRACT

Disclosed are fused dihydro-4H-pyrazolo[5,1-C][1,4]oxazinyl compounds and analog compounds, and pharmaceutically acceptable salts thereof; compositions thereof; and methods of use thereof. These compounds are useful for treating a variety of neurological and psychiatric disorders, such as those described herein.

### CROSS REFERENCE TO RELATED APPLICATIONS

This application claims priority to U.S. Provisional Application No. 63/172,137, filed Apr. 8, 2021, the entire disclosure of which is hereby incorporated herein by reference.

### BACKGROUND

Central nervous system disorders affect a wide range of the population with differing severity. Neurological and psychiatric disorders include major depression, schizophrenia, bipolar disorder, obsessive compulsive disorder (OCD), panic disorder, and posttraumatic stress disorder (PTSD), among others. These disorders affect a person's thoughts, mood, behavior and social interactions and can significantly impair daily functioning. See, e.g., Diagnostic and Statistical Manual of Mental Disorders, 4^{th} Ed., American Psychiatric Association (2000) (“DSM-IV-TR”); Diagnostic and Statistical Manual of Mental Disorders, 5^{th} Ed., American Psychiatric Association (2013) (“DSM-5”).

Bipolar disorder is a serious psychiatric disorder that has a prevalence of approximately 2% of the population, and affects both genders alike. It is a relapsing-remitting condition characterized by cycling between elevated (i.e., manic) and depressed moods, which distinguishes it from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although patients spend considerably more time in the depressive state. Other related conditions include cyclothymic disorder.

Schizophrenia is a psychopathic disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder.

Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the patient suffers from a lack of normal experiences, such as anhedonia and lack of social interaction. The cognitive symptoms relate to cognitive impairment in schizophrenics, such as lack of sustained attention and deficits in decision making.

Neurological and psychiatric disorders can exhibit a variety of symptoms, including cognitive impairment, depressive disorders, and anxiety disorders.

Cognitive impairment includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts.

Depressive disorders include major depressive disorder and dysthymia, and are associated with depressed mood (sadness), poor concentration, insomnia, fatigue, appetite disturbances, excessive guilt and thoughts of suicide.

Anxiety disorders are disorders characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs.

### SUMMARY

While medications exist for some aspects of these diseases, there remains a need for effective treatments for various neurological and psychiatric disorders, including mood disorders such as bipolar and related disorders, psychosis and schizophrenia. For example, while mood stabilizers such as lithium and valproate, antidepressants and antipsychotic drugs are used to treat mood disorders, more effective medications are necessary. And current antipsychotics may be successful in treating the positive symptoms of schizophrenia but fare less well for the negative and cognitive symptoms. Additionally, current antidepressants are typically effective only for a proportion of patients suffering from depression.

In some embodiments, the present invention encompasses the insight that compounds of Table 1 and pharmaceutically acceptable salts thereof, are useful for treating a variety of neurological and psychiatric disorders, such as those described herein:

TABLE 1

| | |
|---|---|
| 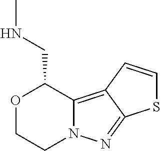 | 1 |
| 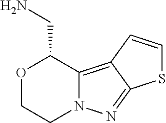 [SMILES: NC[C@H]1OCCn2nc3sccc3c21] | 2 |
| 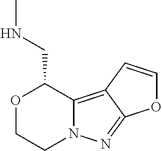 | 3 |
| 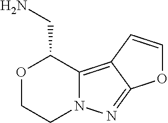 [SMILES: NC[C@H]1OCCn2nc3occc3c21] | 4 |
| 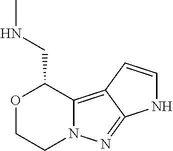 | 5 |
| 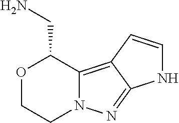 [SMILES: NC[C@H]1OCCn2nc3[nH]ccc3c21] | 6 |
| 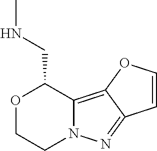 | 7 |
| 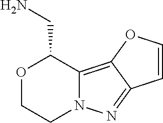 [SMILES: NC[C@H]1OCCn2nc3ccoc3c21] | 8 |
| 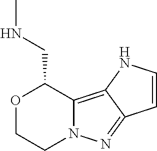 | 9 |
| 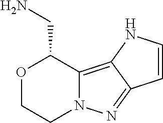 [SMILES: NC[C@H]1OCCn2nc3cc[nH]c3c21] | 10 |
| 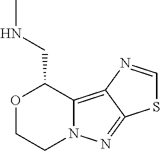 | 11 |
| 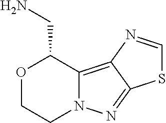 [SMILES: NC[C@H]1OCCn2nc3scnc3c21] | 12 |
| 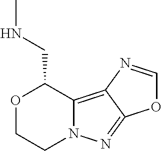 | 13 |
| 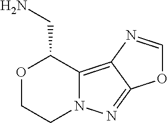 [SMILES: NC[C@H]1OCCn2nc3ocnc3c21] | 14 |
| 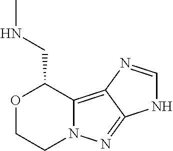 | 15 |
| 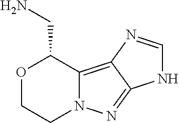 [SMILES: NC[C@H]1OCCn2nc3[nH]cnc3c21] | 16 |
| 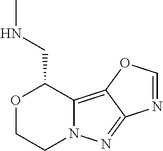 | 17 |
| 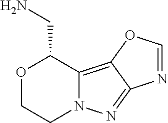 [SMILES: NC[C@H]1OCCn2nc3ncoc3c21] | 18 |
| 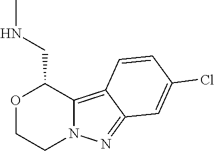 [SMILES: CNC[C@H]1OCCn2nc3cc(Cl)ccc3c21] | 19 |
| 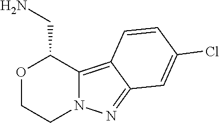 [SMILES: NC[C@H]1OCCn2nc3cc(Cl)ccc3c21] | 20 |
| 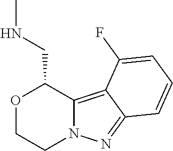 [SMILES: CNC[C@H]1OCCn2nc3cccc(F)c3c21] | 21 |
| 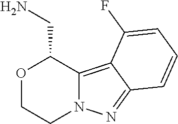 [SMILES: NC[C@H]1OCCn2nc3cccc(F)c3c21] | 22 |
| 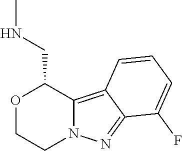 [SMILES: CNC[C@H]1OCCn2nc3c(F)cccc3c21] | 23 |
| 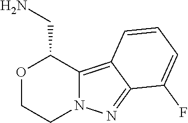 [SMILES: NC[C@H]1OCCn2nc3c(F)cccc3c21] | 24 |
| 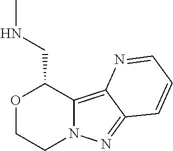 [SMILES: CNC[C@H]1OCCn2nc3cccnc3c21] | 25 |
| 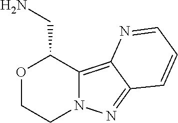 [SMILES: NC[C@H]1OCCn2nc3cccnc3c21] | 26 |
| 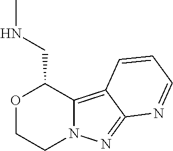 [SMILES: CNC[C@H]1OCCn2nc3ncccc3c21] | 27 |
| 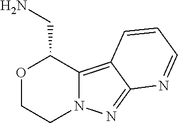 [SMILES: NC[C@H]1OCCn2nc3ncccc3c21] | 28 |
| 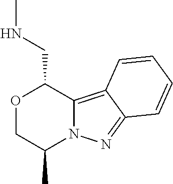 [SMILES: CNC[C@H]1OC[C@H](C)n2nc3ccccc3c21] | 29 |
| 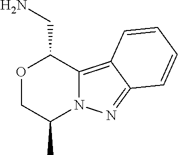 [SMILES: C[C@H]1CO[C@H](CN)c2c3ccccc3nn21] | 30 |
| 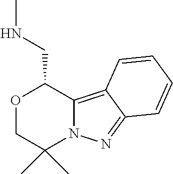 [SMILES: CNC[C@H]1OCC(C)(C)n2nc3ccccc3c21] | 31 |
| 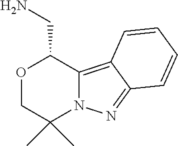 [SMILES: CC1(C)CO[C@H](CN)c2c3ccccc3nn21] | 32 |
| 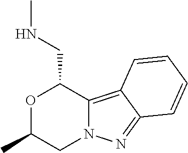 [SMILES: CNC[C@H]1O[C@H](C)Cn2nc3ccccc3c21] | 33 |
| 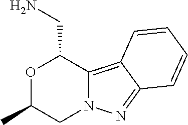 [SMILES: C[C@@H]1Cn2nc3ccccc3c2[C@@H](CN)O1] | 34 |
| 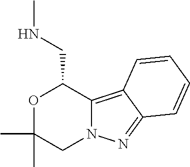 [SMILES: CNC[C@H]1OC(C)(C)Cn2nc3ccccc3c21] | 35 |
| 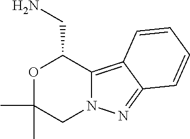 [SMILES: CC1(C)Cn2nc3ccccc3c2[C@@H](CN)O1] | 36 |
| 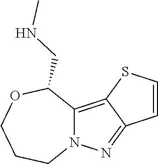 [SMILES: CNC[C@H]1OCCCn2nc3ccsc3c21] | 37 |
| 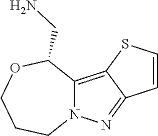 [SMILES: NC[C@H]1OCCCn2nc3ccsc3c21] | 38 |
| 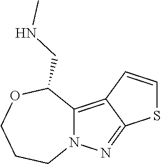 [SMILES: CNC[C@H]1OCCCn2nc3sccc3c21] | 39 |
| 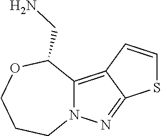 [SMILES: NC[C@H]1OCCCn2nc3sccc3c21] | 40 |
| 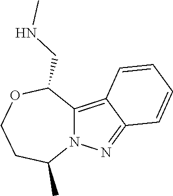 [SMILES: CNC[C@H]1OCC[C@H](C)n2nc3ccccc3c21] | 41 |
| 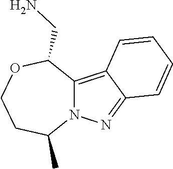 [SMILES: C[C@H]1CCO[C@H](CN)c2c3ccccc3nn21] | 42 |
| 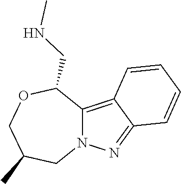 [SMILES: CNC[C@H]1OC[C@H](C)Cn2nc3ccccc3c21] | 43 |
| 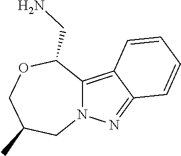 [SMILES: C[C@H]1CO[C@H](CN)c2c3ccccc3nn2C1] | 44 |
| 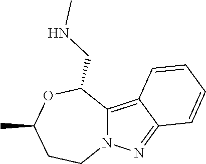 [SMILES: CNC[C@H]1O[C@H](C)CCn2nc3ccccc3c21] | 45 |
| 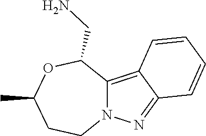 [SMILES: C[C@@H]1CCn2nc3ccccc3c2[C@@H](CN)O1] | 46 |
| 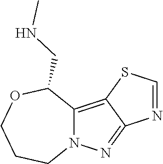 [SMILES: CNC[C@H]1OCCCn2nc3ncsc3c21] | 47 |
| 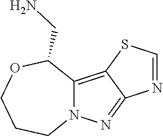 [SMILES: NC[C@H]1OCCCn2nc3ncsc3c21] | 48 |
| 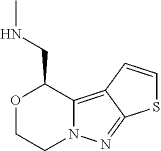 | 49 |
| 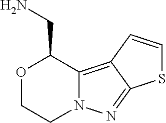 [SMILES: NC[C@@H]1OCCn2nc3sccc3c21] | 50 |
| 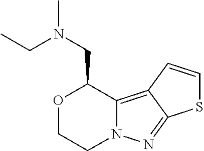 [SMILES: CCN(C)C[C@@H]1OCCn2nc3sccc3c21] | 51 |
| 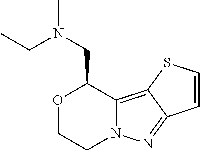 [SMILES: CCN(C)C[C@@H]1OCCn2nc3ccsc3c21] | 52 |
| 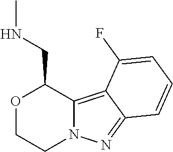 [SMILES: CNC[C@@H]1OCCn2nc3cccc(F)c3c21] | 53 |
| 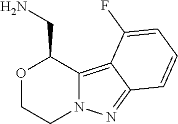 [SMILES: NC[C@@H]1OCCn2nc3cccc(F)c3c21] | 54 |
| 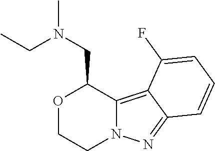 [SMILES: CCN(C)C[C@@H]1OCCn2nc3cccc(F)c3c21] | 55 |
| 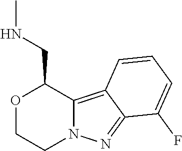 [SMILES: CNC[C@@H]1OCCn2nc3c(F)cccc3c21] | 56 |
| 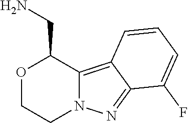 [SMILES: NC[C@@H]1OCCn2nc3c(F)cccc3c21] | 57 |
| 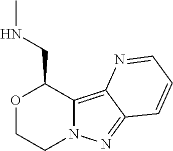 [SMILES: CNC[C@@H]1OCCn2nc3cccnc3c21] | 58 |
| 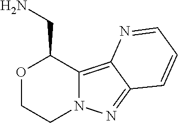 [SMILES: NC[C@@H]1OCCn2nc3cccnc3c21] | 59 |
| 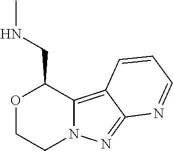 [SMILES: CNC[C@@H]1OCCn2nc3ncccc3c21] | 60 |
| 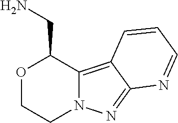 [SMILES: NC[C@@H]1OCCn2nc3ncccc3c21] | 61 |
| 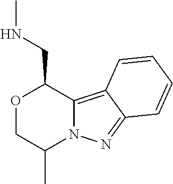 [SMILES: CNC[C@@H]1OCC(C)n2nc3ccccc3c21] | 62 |
| 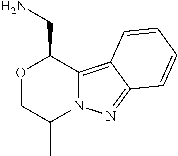 [SMILES: CC1CO[C@@H](CN)c2c3ccccc3nn21] | 63 |
| 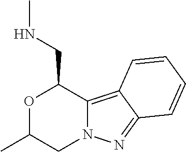 [SMILES: CNC[C@@H]1OC(C)Cn2nc3ccccc3c21] | 64 |
| 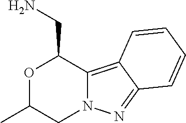 [SMILES: CC1Cn2nc3ccccc3c2[C@H](CN)O1] | 65 |
| 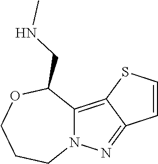 [SMILES: CNC[C@@H]1OCCCn2nc3ccsc3c21] | 66 |
| 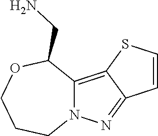 [SMILES: NC[C@@H]1OCCCn2nc3ccsc3c21] | 67 |
| 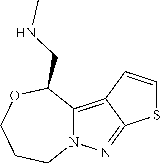 [SMILES: CNC[C@@H]1OCCCn2nc3sccc3c21] | 68 |
| 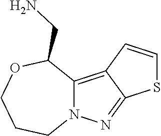 [SMILES: NC[C@@H]1OCCCn2nc3sccc3c21] | 69 |

Also provided herein are methods for the treatment of various neurological and psychiatric disorders using the compounds and compositions provided herein.

### DETAILED DESCRIPTION OF CERTAIN EMBODIMENTS

### 1. General Description of Compounds of the Invention

In some embodiments, the present invention provides a compound shown in Table 1, or a pharmaceutically acceptable salt thereof:

TABLE 1

| | |
|---|---|
| 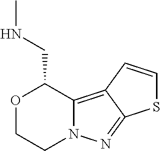 | 1 |
| 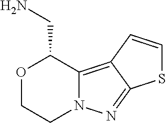 [SMILES: NC[C@H]1OCCn2nc3sccc3c21] | 2 |
| 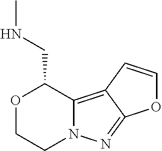 | 3 |
| 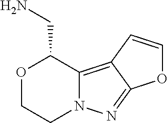 [SMILES: NC[C@H]1OCCn2nc3occc3c21] | 4 |
| 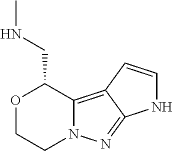 | 5 |
| 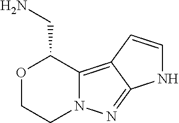 [SMILES: NC[C@H]1OCCn2nc3[nH]ccc3c21] | 6 |
| 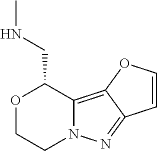 | 7 |
| 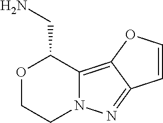 [SMILES: NC[C@H]1OCCn2nc3ccoc3c21] | 8 |
| 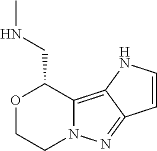 | 9 |
| 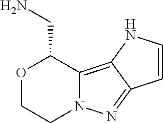 [SMILES: NC[C@H]1OCCn2nc3cc[nH]c3c21] | 10 |
| 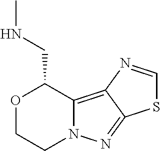 | 11 |
| 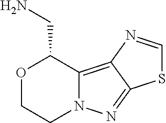 [SMILES: NC[C@H]1OCCn2nc3scnc3c21] | 12 |
| 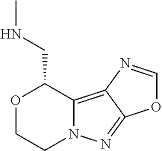 | 13 |
| 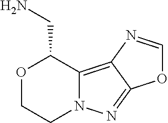 [SMILES: NC[C@H]1OCCn2nc3ocnc3c21] | 14 |
| 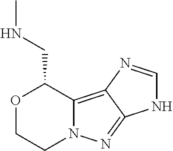 | 15 |
| 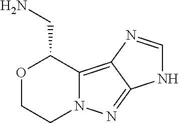 [SMILES: NC[C@H]1OCCn2nc3[nH]cnc3c21] | 16 |
| 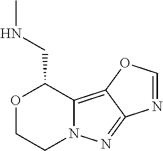 | 17 |
| 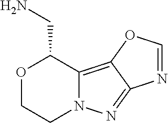 [SMILES: NC[C@H]1OCCn2nc3ncoc3c21] | 18 |
| 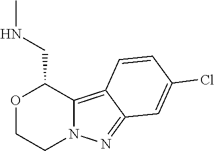 [SMILES: CNC[C@H]1OCCn2nc3cc(Cl)ccc3c21] | 19 |
| 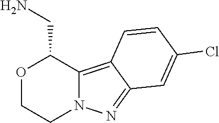 [SMILES: NC[C@H]1OCCn2nc3cc(Cl)ccc3c21] | 20 |
| 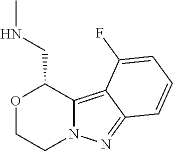 [SMILES: CNC[C@H]1OCCn2nc3cccc(F)c3c21] | 21 |
| 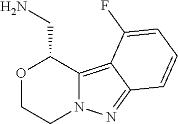 [SMILES: NC[C@H]1OCCn2nc3cccc(F)c3c21] | 22 |
| 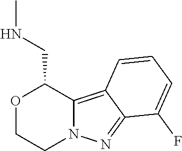 [SMILES: CNC[C@H]1OCCn2nc3c(F)cccc3c21] | 23 |
| 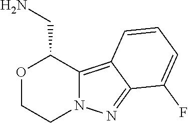 [SMILES: NC[C@H]1OCCn2nc3c(F)cccc3c21] | 24 |
| 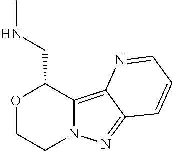 [SMILES: CNC[C@H]1OCCn2nc3cccnc3c21] | 25 |
| 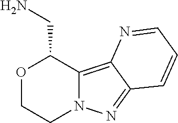 [SMILES: NC[C@H]1OCCn2nc3cccnc3c21] | 26 |
| 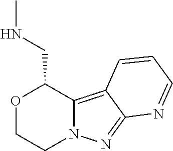 [SMILES: CNC[C@H]1OCCn2nc3ncccc3c21] | 27 |
| 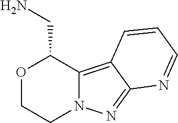 [SMILES: NC[C@H]1OCCn2nc3ncccc3c21] | 28 |
| 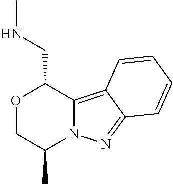 [SMILES: CNC[C@H]1OC[C@H](C)n2nc3ccccc3c21] | 29 |
| 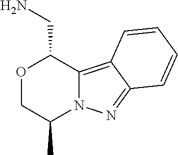 [SMILES: C[C@H]1CO[C@H](CN)c2c3ccccc3nn21] | 30 |
| 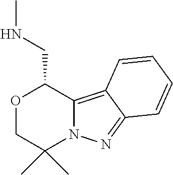 [SMILES: CNC[C@H]1OCC(C)(C)n2nc3ccccc3c21] | 31 |
| 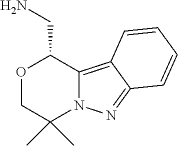 [SMILES: CC1(C)CO[C@H](CN)c2c3ccccc3nn21] | 32 |
| 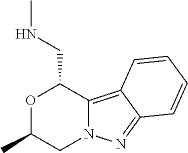 [SMILES: CNC[C@H]1O[C@H](C)Cn2nc3ccccc3c21] | 33 |
| 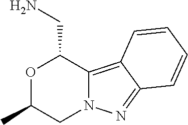 [SMILES: C[C@@H]1Cn2nc3ccccc3c2[C@@H](CN)O1] | 34 |
| 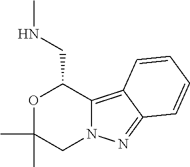 [SMILES: CNC[C@H]1OC(C)(C)Cn2nc3ccccc3c21] | 35 |
| 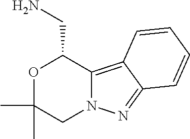 [SMILES: CC1(C)Cn2nc3ccccc3c2[C@@H](CN)O1] | 36 |
| 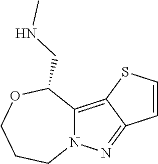 [SMILES: CNC[C@H]1OCCCn2nc3ccsc3c21] | 37 |
| 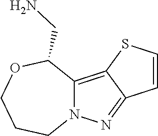 [SMILES: NC[C@H]1OCCCn2nc3ccsc3c21] | 38 |
| 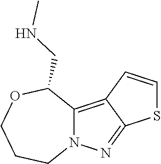 [SMILES: CNC[C@H]1OCCCn2nc3sccc3c21] | 39 |
| 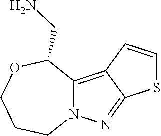 [SMILES: NC[C@H]1OCCCn2nc3sccc3c21] | 40 |
| 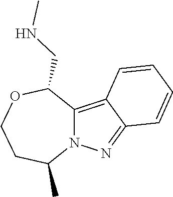 [SMILES: CNC[C@H]1OCC[C@H](C)n2nc3ccccc3c21] | 41 |
| 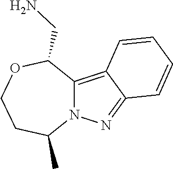 [SMILES: C[C@H]1CCO[C@H](CN)c2c3ccccc3nn21] | 42 |
| 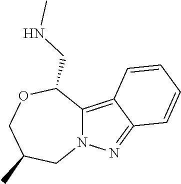 [SMILES: CNC[C@H]1OC[C@H](C)Cn2nc3ccccc3c21] | 43 |
| 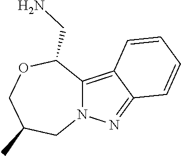 [SMILES: C[C@H]1CO[C@H](CN)c2c3ccccc3nn2C1] | 44 |
| 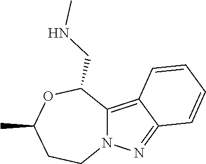 [SMILES: CNC[C@H]1O[C@H](C)CCn2nc3ccccc3c21] | 45 |
| 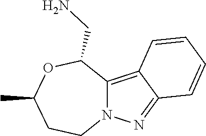 [SMILES: C[C@@H]1CCn2nc3ccccc3c2[C@@H](CN)O1] | 46 |
| 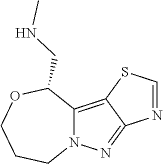 [SMILES: CNC[C@H]1OCCCn2nc3ncsc3c21] | 47 |
| 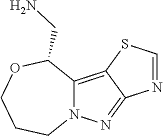 [SMILES: NC[C@H]1OCCCn2nc3ncsc3c21] | 48 |
| 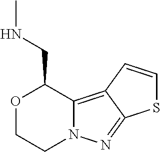 | 49 |
| 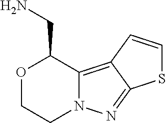 [SMILES: NC[C@@H]1OCCn2nc3sccc3c21] | 50 |
| 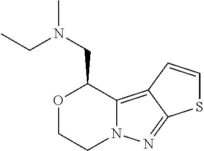 [SMILES: CCN(C)C[C@@H]1OCCn2nc3sccc3c21] | 51 |
| 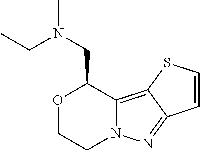 [SMILES: CCN(C)C[C@@H]1OCCn2nc3ccsc3c21] | 52 |
| 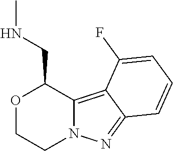 [SMILES: CNC[C@@H]1OCCn2nc3cccc(F)c3c21] | 53 |
| 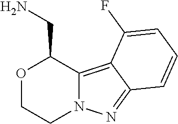 [SMILES: NC[C@@H]1OCCn2nc3cccc(F)c3c21] | 54 |
| 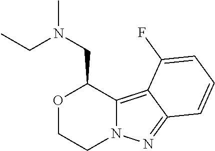 [SMILES: CCN(C)C[C@@H]1OCCn2nc3cccc(F)c3c21] | 55 |
| 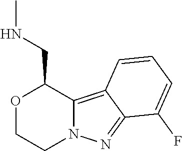 [SMILES: CNC[C@@H]1OCCn2nc3c(F)cccc3c21] | 56 |
| 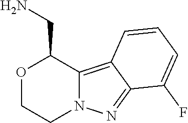 [SMILES: NC[C@@H]1OCCn2nc3c(F)cccc3c21] | 57 |
| 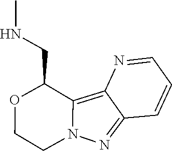 [SMILES: CNC[C@@H]1OCCn2nc3cccnc3c21] | 58 |
| 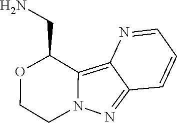 [SMILES: NC[C@@H]1OCCn2nc3cccnc3c21] | 59 |
| 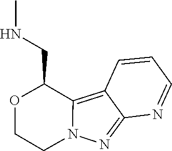 [SMILES: CNC[C@@H]1OCCn2nc3ncccc3c21] | 60 |
| 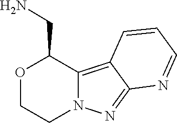 [SMILES: NC[C@@H]1OCCn2nc3ncccc3c21] | 61 |
| 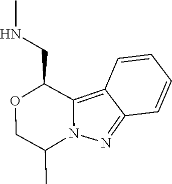 [SMILES: CNC[C@@H]1OCC(C)n2nc3ccccc3c21] | 62 |
| 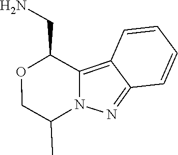 [SMILES: CC1CO[C@@H](CN)c2c3ccccc3nn21] | 63 |
| 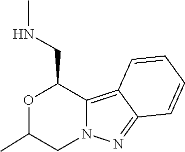 [SMILES: CNC[C@@H]1OC(C)Cn2nc3ccccc3c21] | 64 |
| 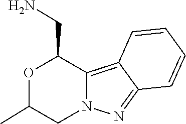 [SMILES: CC1Cn2nc3ccccc3c2[C@H](CN)O1] | 65 |
| 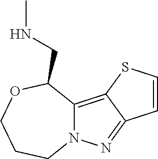 [SMILES: CNC[C@@H]1OCCCn2nc3ccsc3c21] | 66 |
| 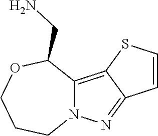 [SMILES: NC[C@@H]1OCCCn2nc3ccsc3c21] | 67 |
| 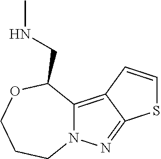 [SMILES: CNC[C@@H]1OCCCn2nc3sccc3c21] | 68 |
| 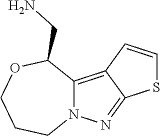 [SMILES: NC[C@@H]1OCCCn2nc3sccc3c21] | 69 |

### 2. Compounds and Definitions

Compounds of this invention include those described generally above, and are further illustrated by the species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in M. Loudon, Organic Chemistry, 5th Ed., Roberts and Company, Greenwood Village, CO: 2009; and M. B. Smith, March's Advanced Organic Chemistry: Reactions, Mechanisms and Structure, 7th Ed., John Wiley & Sons, Hoboken: 2013, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Unless otherwise specified, the word “includes” (or any variation thereon, e.g., “include”, “including”, etc.) is intended to be open-ended (and not limited to the examples cited in the text following “includes”). For example, the phrase—“parenteral, including . . . ” as used herein to describe one route of administering a medicament contemplates any route of administering a medicament via injection or infusion, using, for example, but not limited to, a syringe or catheter, which techniques include but are not limited to administration via subcutaneous, intravenous, intramuscular, intra-articular, intra-synovial, intrasternal, intrathecal, intrahepatic, intralesional and intracranial injection or infusion techniques.

Unless otherwise specified, the phrase “such as” is intended to be open-ended. For example, “mood disorders such as bipolar and related disorders, psychosis and schizophrenia” means that a mood disorder can be, but is not limited to, bipolar and related disorders, psychosis and schizophrenia.

Where one or more ranges are referred to throughout this specification, each range is understood to encompass each discrete point within the range, including the endpoints describing the range, as if the same were fully set forth herein.

### 3. Description of Exemplary Embodiments

Exemplary compounds of formula (I) are set forth in Table 1, below.

TABLE 1

| | |
|---|---|
| 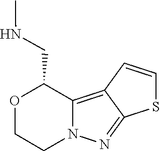 | 1 |
| 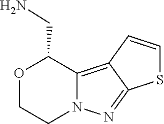 [SMILES: NC[C@H]1OCCn2nc3sccc3c21] | 2 |
| 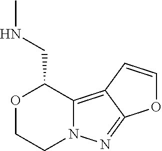 | 3 |
| 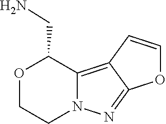 [SMILES: NC[C@H]1OCCn2nc3occc3c21] | 4 |
| 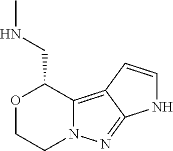 | 5 |
| 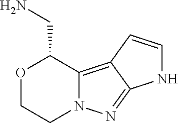 [SMILES: NC[C@H]1OCCn2nc3[nH]ccc3c21] | 6 |
| 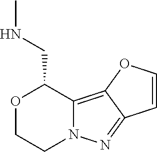 | 7 |
| 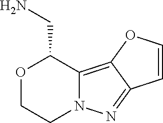 [SMILES: NC[C@H]1OCCn2nc3ccoc3c21] | 8 |
| 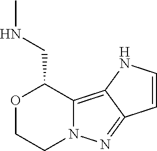 | 9 |
| 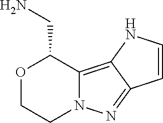 [SMILES: NC[C@H]1OCCn2nc3cc[nH]c3c21] | 10 |
| 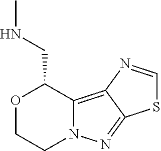 | 11 |
| 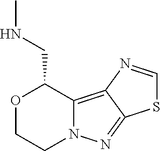 | 12 |
| 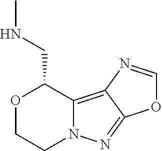 | 13 |
| 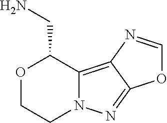 [SMILES: NC[C@H]1OCCn2nc3ocnc3c21] | 14 |
| 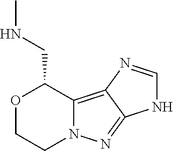 | 15 |
| 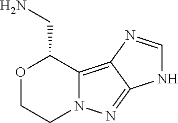 [SMILES: NC[C@H]1OCCn2nc3[nH]cnc3c21] | 16 |
| 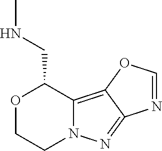 | 17 |
| 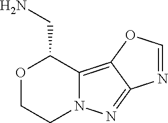 [SMILES: NC[C@H]1OCCn2nc3ncoc3c21] | 18 |
| 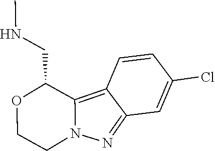 [SMILES: CNC[C@H]1OCCn2nc3cc(Cl)ccc3c21] | 19 |
| 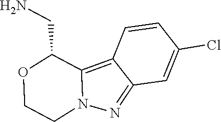 [SMILES: NC[C@H]1OCCn2nc3cc(Cl)ccc3c21] | 20 |
| 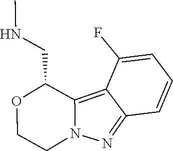 [SMILES: CNC[C@H]1OCCn2nc3cccc(F)c3c21] | 21 |
| 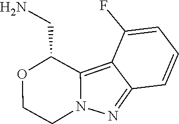 [SMILES: NC[C@H]1OCCn2nc3cccc(F)c3c21] | 22 |
| 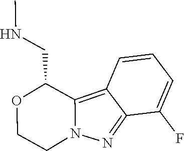 [SMILES: CNC[C@H]1OCCn2nc3c(F)cccc3c21] | 23 |
| 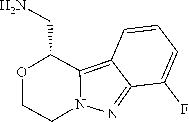 [SMILES: NC[C@H]1OCCn2nc3c(F)cccc3c21] | 24 |
| 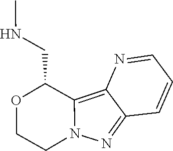 [SMILES: CNC[C@H]1OCCn2nc3cccnc3c21] | 25 |
| 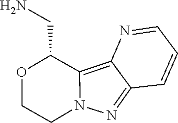 [SMILES: NC[C@H]1OCCn2nc3cccnc3c21] | 26 |
| 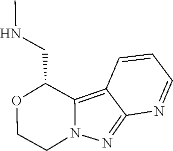 [SMILES: CNC[C@H]1OCCn2nc3ncccc3c21] | 27 |
| 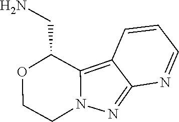 [SMILES: NC[C@H]1OCCn2nc3ncccc3c21] | 28 |
| 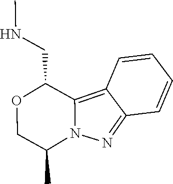 [SMILES: CNC[C@H]1OC[C@H](C)n2nc3ccccc3c21] | 29 |
| 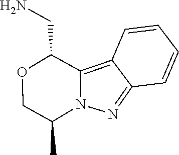 [SMILES: C[C@H]1CO[C@H](CN)c2c3ccccc3nn21] | 30 |
| 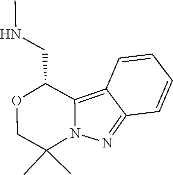 [SMILES: CNC[C@H]1OCC(C)(C)n2nc3ccccc3c21] | 31 |
| 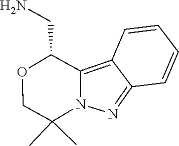 [SMILES: CC1(C)CO[C@H](CN)c2c3ccccc3nn21] | 32 |
| 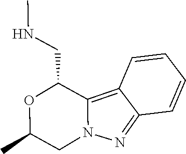 [SMILES: CNC[C@H]1O[C@H](C)Cn2nc3ccccc3c21] | 33 |
| 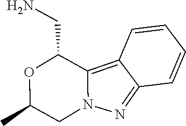 [SMILES: C[C@@H]1Cn2nc3ccccc3c2[C@@H](CN)O1] | 34 |
| 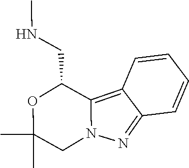 [SMILES: CNC[C@H]1OC(C)(C)Cn2nc3ccccc3c21] | 35 |
| 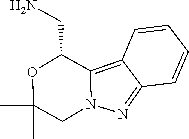 [SMILES: CC1(C)Cn2nc3ccccc3c2[C@@H](CN)O1] | 36 |
| 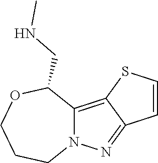 [SMILES: CNC[C@H]1OCCCn2nc3ccsc3c21] | 37 |
| 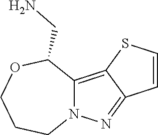 [SMILES: NC[C@H]1OCCCn2nc3ccsc3c21] | 38 |
| 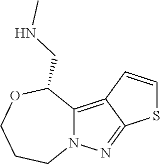 [SMILES: CNC[C@H]1OCCCn2nc3sccc3c21] | 39 |
| 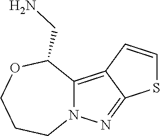 [SMILES: NC[C@H]1OCCCn2nc3sccc3c21] | 40 |
| 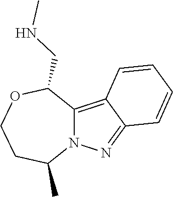 [SMILES: CNC[C@H]1OCC[C@H](C)n2nc3ccccc3c21] | 41 |
| 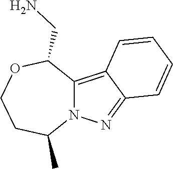 [SMILES: C[C@H]1CCO[C@H](CN)c2c3ccccc3nn21] | 42 |
| 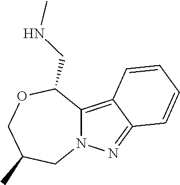 [SMILES: CNC[C@H]1OC[C@H](C)Cn2nc3ccccc3c21] | 43 |
| 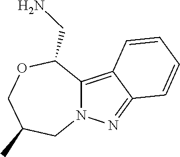 [SMILES: C[C@H]1CO[C@H](CN)c2c3ccccc3nn2C1] | 44 |
| 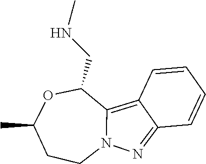 [SMILES: CNC[C@H]1O[C@H](C)CCn2nc3ccccc3c21] | 45 |
| 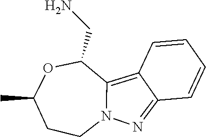 [SMILES: C[C@@H]1CCn2nc3ccccc3c2[C@@H](CN)O1] | 46 |
| 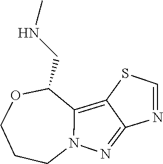 [SMILES: CNC[C@H]1OCCCn2nc3ncsc3c21] | 47 |
| 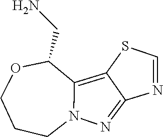 [SMILES: NC[C@H]1OCCCn2nc3ncsc3c21] | 48 |
| 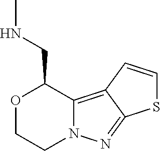 | 49 |
| 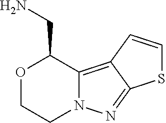 [SMILES: NC[C@@H]1OCCn2nc3sccc3c21] | 50 |
| 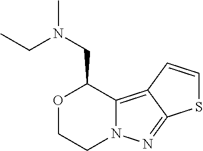 [SMILES: CCN(C)C[C@@H]1OCCn2nc3sccc3c21] | 51 |
| 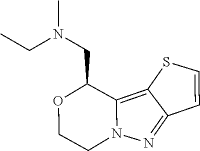 [SMILES: CCN(C)C[C@@H]1OCCn2nc3ccsc3c21] | 52 |
| 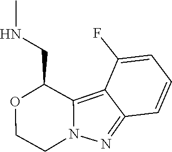 [SMILES: CNC[C@@H]1OCCn2nc3cccc(F)c3c21] | 53 |
| 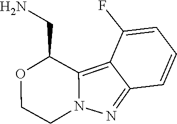 [SMILES: NC[C@@H]1OCCn2nc3cccc(F)c3c21] | 54 |
| 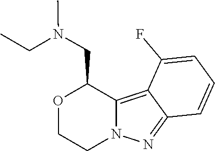 [SMILES: CCN(C)C[C@@H]1OCCn2nc3cccc(F)c3c21] | 55 |
| 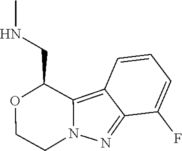 [SMILES: CNC[C@@H]1OCCn2nc3c(F)cccc3c21] | 56 |
| 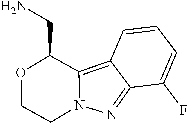 [SMILES: NC[C@@H]1OCCn2nc3c(F)cccc3c21] | 57 |
| 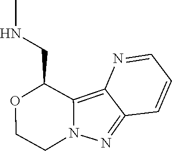 [SMILES: CNC[C@@H]1OCCn2nc3cccnc3c21] | 58 |
| 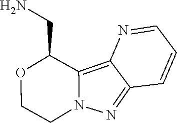 [SMILES: NC[C@@H]1OCCn2nc3cccnc3c21] | 59 |
| 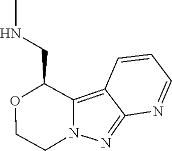 [SMILES: CNC[C@@H]1OCCn2nc3ncccc3c21] | 60 |
| 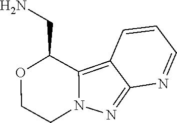 [SMILES: NC[C@@H]1OCCn2nc3ncccc3c21] | 61 |
| 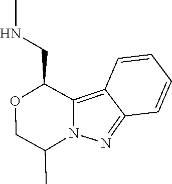 [SMILES: CNC[C@@H]1OCC(C)n2nc3ccccc3c21] | 62 |
| 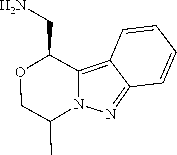 [SMILES: CC1CO[C@@H](CN)c2c3ccccc3nn21] | 63 |
| 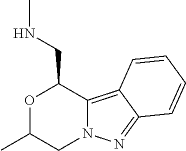 [SMILES: CNC[C@@H]1OC(C)Cn2nc3ccccc3c21] | 64 |
| 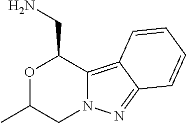 [SMILES: CC1Cn2nc3ccccc3c2[C@H](CN)O1] | 65 |
| 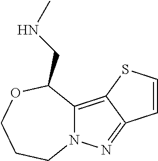 [SMILES: CNC[C@@H]1OCCCn2nc3ccsc3c21] | 66 |
| 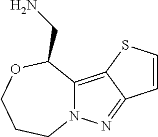 [SMILES: NC[C@@H]1OCCCn2nc3ccsc3c21] | 67 |
| 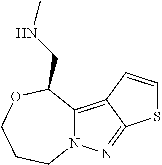 [SMILES: CNC[C@@H]1OCCCn2nc3sccc3c21] | 68 |
| 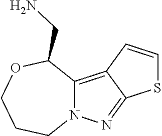 [SMILES: NC[C@@H]1OCCCn2nc3sccc3c21] | 69 |

In some embodiments, the present invention provides a compound selected from those depicted in Table 1, above, or a pharmaceutically acceptable salt thereof In some embodiments, the pharmaceutically acceptable salt is a hydrochloride salt.

### 4. Uses, Formulation and Administration and Pharmaceutically Acceptable Compositions

According to another embodiment, the invention provides a composition comprising a compound of this invention, or a pharmaceutically acceptable salt, ester, or salt of ester thereof, and a pharmaceutically acceptable carrier, adjuvant, or vehicle. In some embodiments, the amount of compound in compositions of this invention is such that is effective to treat, prevent, and/or manage various neurological and/or psychiatric disorders and/or symptoms in a patient. In some embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon a variety of factors, including the host treated and the particular mode of administration. Preferably, provided compositions should be formulated so that a dosage of between 0.01-100 mg/kg body weight/day of a compound of the present invention can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### 5. Uses of Compounds and Pharmaceutically Acceptable Compositions

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds and compositions, according to the method of the present invention, may be administered using any amount and any route of administration effective for treating a neurological or psychiatric disorder.

In some embodiments, the compounds and compositions, according to the method of the present invention, may be administered using any amount and any route of administration effective for treating a neurological and/or psychiatric disorder in a patient.

In some embodiments, the neurological or psychiatric disorder is selected from a psychosis, including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosis, psychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Down syndrome, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, Parkinsonism-ALS dementia complex and basal ganglia calcification), medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias {including drug e.g. L-DOPA induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics), and dystonia (including generalized dystonia such as idiopathic dystonia, drug-induced dystonia, symptomatic dystonia and paroxsymal dystonia, and focal dystonia such as blepharospasm, oromandibular dystonia, spasmodic dysphonia, spasmodic torticollis, axial dystonia, dystonic writer's cramp and hemiplegic dystonia)}; urinary incontinence; neuronal damage including ocular damage, retinopathy or macular degeneration of the eye, tinnitus, hearing impairment and loss, and brain edema; emesis; and sleep disorders including insomnia and narcolepsy.

In some embodiments, the neurological or psychiatric disorder is Alzheimer's Disease, Parkinson's Disease, depression, cognitive impairment, stroke, schizophrenia, Down Syndrome, or Fetal Alcohol Syndrome. In some embodiments, the neurological or psychiatric disorder is Alzheimer's Disease. In some embodiments, the neurological or psychiatric disorder is Parkinson's Disease. In some embodiments, the neurological or psychiatric disorder is depression. In some embodiments, the neurological or psychiatric disorder is cognitive impairment. In some embodiments, the cognitive impairment is cognitive dysfunction associated with depression, for example, major depressive disorder. In some embodiments, the neurological or psychiatric disorder is stroke. In some embodiments, the neurological or psychiatric disorder is schizophrenia. In some embodiments, the neurological or psychiatric disorder is Down Syndrome. In some embodiments, the neurological or psychiatric disorder is Fetal Alcohol Syndrome.

In some embodiments, the neurological or psychiatric disorder involves a deficit in cognition (cognitive domains as defined by the DSM-5 are: complex attention, executive function, learning and memory, language, perceptual-motor, social cognition). In some embodiments, the neurological or psychiatric disorder is associated with a deficit in dopamine signaling. In some embodiments, the neurological or psychiatric disorder is associated with basal ganglia dysfunction. In some embodiments, the neurological or psychiatric disorder is associated with dysregulated locomotor activity. In some embodiments, the neurological or psychiatric disorder is associated with impairment of prefrontal cortex functioning.

In some embodiments, the present invention provides a method of treating one or more symptoms of a neurological and/or psychiatric disorder provided herein. Such disorders include mood disorders, including bipolar I disorder, bipolar II disorder, bipolar depression, mania, cyclothymic disorder, substance/medication-induced bipolar and related disorders, bipolar and related disorder due to another medical condition, other specified bipolar and related disorder, and unspecified bipolar and related disorders; psychotic disorders, including schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), schizoaffective disorder, aggression, delirium, Parkinson's psychosis, excitative psychosis, Tourette's syndrome, and organic or NOS psychosis; depressive disorders, including disruptive mood dysregulation disorder, major depressive disorder (MDD) (including major depressive episode), dysthymia, persistent depressive disorder (dysthymia), treatment resistant depression, premenstrual dysphoric disorder, substance/medication-induced depressive disorder, depressive disorder due to another medical condition, other specified depressive disorder, and unspecified depressive disorder; anxiety disorders, including separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders; and other disorders including substance abuse or dependency (e.g., nicotine, alcohol, cocaine), addiction, eating disorders, behavior disorder, seizure, vertigo, epilepsy, agitation, aggression, neurodegenerative disease, Alzheimer's disease, Parkinson's disease, dyskinesias, Huntington's disease, dementia, premenstrual dysphoria; and attention deficit disorder (ADD) and neurodevelopmental disorders, including attention deficit hyperactivity disorder (ADHD)), autism, autism spectrum disorder, obsessive-compulsive disorder, pain (e.g., neuropathic pain, sensitization accompanying neuropathic pain, and inflammatory pain), fibromyalgia, migraine, cognitive impairment, movement disorder, restless leg syndrome (RLS), multiple sclerosis, Parkinson's disease, Huntington's disease, dyskinesias multiple sclerosis, sleep disorder, sleep apnea, narcolepsy, excessive daytime sleepiness, jet lag, drowsy side effect of medications, insomnia, sexual dysfunction, hypertension, emesis, Lesche-Nyhane disease, Wilson's disease, and Huntington's chorea. In some embodiments, the neurological and/or psychiatric disorders include agitation and aggression. In some embodiments, the agitation and aggression are associated with Alzheimer's Disease, Parkinson's Disease, and/or autism. In some embodiments, the neurological and/or psychiatric disorders are obsessive-compulsive disorder and related disorders (e.g., body dysmorphic disorder, hoarding disorder, trichotillomania, excoriation disorder). In some embodiments, the neurological and/or psychiatric disorders are disruptive, impulse-control, and conduct disorders including oppositional defiant disorder, intermittent explosive disorder, conduct disorder, antisocial personality disorder, pyromania, kleptomania, other specified disruptive, impulse-control, and conduct disorder, unspecified disruptive, impulse-control, and conduct disorder.

In some embodiments, the present invention provides a method of treating one or more symptoms including depression (e.g., major depressive disorder or dysthymia); bipolar disorder, seasonal affective disorder; cognitive deficit; sleep related disorder (e.g., sleep apnea, insomnia, narcolepsy, cataplexy) including those sleep disorders which are produced by psychiatric conditions; chronic fatigue syndrome; anxieties (e.g., general anxiety disorder, social anxiety disorder, panic disorder); obsessive compulsive disorder; post-menopausal vasomotor symptoms (e.g., hot flashes, night sweats); neurodegenerative disease (e.g., Parkinson's disease, Alzheimer's disease and amyotrophic lateral sclerosis); manic disorder; dysthymic disorder; and obesity.

In some embodiments, a depressive disorder is associated with acute suicidality or suicide ideation. The United States Food and Drug Administration has adopted a “black box” label warning indicating that antidepressants may increase the risk of suicidal thinking and behavior in some children, adolescents and young adults (up to age 24) with a depressive disorder such as MDD. In some embodiments, a provided compound does not increase the risk of suicidal thinking and/or behavior in children, adolescents and/or young adults with a depressive disorder, e.g., with MDD. In some embodiments, the present invention provides a method of treating one or more symptoms of a depressive disorder (e.g., MDD) in children, adolescents and/or young adults without increasing the risk of suicidal thinking and/or behavior.

In some embodiments, the present invention provides a method of treating one or more symptoms including senile dementia, Alzheimer's type dementia, cognition, memory loss, amnesia/amnestic syndrome, disturbances of consciousness, coma, lowering of attention, speech disorder, Lennox syndrome, and hyperkinetic syndrome.

In some embodiments, the present invention provides a method of treating one or more symptoms of neuropathic pain, including post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use).

In some embodiments, the present invention provides a method of treating one or more symptoms including obesity; migraine or migraine headache; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In some embodiments, the present invention provides a method of suppressing rapid eye movement (REM) during both sleep and daytime equivalent.

In some embodiments, the present invention provides a method of suppressing or eliminating pathological or excessive REM during the night or daytime equivalent.

In some embodiments, the present invention provides a method of treating one or more symptoms including cataplexy (sudden involuntary transient bouts of muscle weakness or paralysis while awake); nighttime sleep disturbance/sleep fragmentation associated with narcolepsy or other conditions; sleep paralysis associated with narcolepsy or other conditions; hypnagogic and hypnopompic hallucinations associated with narcolepsy or other conditions; and excessive daytime sleepiness associated with narcolepsy, sleep apnea or shift work disorder and other medical conditions such as cancer, chronic fatigue syndrome and fibromyalgia.

In some embodiments, the present invention provides a method of treating a neurological and/or psychiatric disorder described herein, comprising administering a compound of the invention in conjunction with one or more pharmaceutical agents. Suitable pharmaceutical agents that may be used in combination with the compounds of the present invention include anti-Parkinson's drugs, anti-Alzheimer's drugs, anti-depressants, anti-psychotics, anti-ischemics, central nervous system (CNS) depressants, anti-cholinergics, and nootropics. In some embodiments, suitable pharmaceutical agents are anxiolytics.

Suitable anti-Parkinson's drugs include dopamine replacement therapy (e.g., L-DOPA, carbidopa, COMT inhibitors such as entacapone), dopamine agonists (e.g., D1 agonists, D2 agonists, mixed D1/D2 agonists; bromocriptine, pergolide, cabergoline, ropinirole, pramipexole, or apomorphine in combination with domperidone), histamine H2 antagonists, and monoamine oxidase inhibitors such as selegiline and tranylcypromine.

In some embodiments, compounds of the invention can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include beta-secretase inhibitors, gamma-secretase inhibitors, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof. In some embodiments, suitable anti-depressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, chlorpromazine, perphenazine, thioridazine, trifluoperazine, aripiprazole, asenapine, clozapine, olanzapine, paliperidone, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid and pharmaceutically acceptable salts thereof.

In some embodiments, compounds of the invention may be used in combination with other therapies. Suitable therapies include psychotherapy, cognitive behavioral therapy, electroconvulsive therapy, transcranial magnetic stimulation, vagus nerve stimulation, and deep-brain stimulation.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In some embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In some embodiments, a combination of 2 or more therapeutic agents may be administered together with the compounds of the invention. In some embodiments, a combination of 3 or more therapeutic agents may be administered with the compounds of the invention.

Other examples of agents the compounds of this invention may also be combined with include: vitamins and nutritional supplements, antiemetics (e.g. 5 -HT3 receptor antagonists, dopamine antagonists, NK1 receptor antagonists, histamine receptor antagonists, cannabinoids, benzodiazepines, or anticholinergics), agents for treating Multiple Sclerosis (MS) such as beta interferon (e.g., Avonex® and Rebif®), Copaxone®, and mitoxantrone; treatments for asthma such as albuterol and Singulair®; anti-inflammatory agents such as corticosteroids, TNF blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In some embodiments, a compound of the present invention, or a pharmaceutically acceptable salt thereof, is administered in combination with an antisense agent, a monoclonal or polyclonal antibody, or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I, or a pharmaceutically acceptable salt thereof, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 mg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present invention provides a medicament comprising at least one compound of formula I, or a pharmaceutically acceptable salt thereof, and a pharmaceutically acceptable carrier, adjuvant, or vehicle.

In some embodiments, the present invention provides the use of a compound of formula I, or a pharmaceutically acceptable salt thereof, in the manufacture of a medicament for the treatment of a neurological and/or psychiatric disorder.

### EXAMPLES

### Example 1. Syntheses

As depicted in the Examples below, in some embodiments, compounds are prepared according to the following procedures. It will be appreciated that, although the general methods depict the synthesis of certain compounds of the present invention, the following methods, and other methods known to persons skilled in the art, can be applied to all compounds and subclasses and species of each of these, as described herein.

### General Schemes

Schemes below provide exemplary synthetic methods for the preparation of the compounds provided herein. One of ordinary skills in the art will understand that similar methods may be employed to prepare the compounds provided herein. In other words, one of ordinary skills in the art will recognize that suitable adjustments to reagents, protecting groups, reaction conditions, reaction sequences, purification methods, and chiral separation conditions may be employed to prepare a desired embodiment. The reactions may be scaled upwards or downwards to suit the amount of material to be prepared.

In one embodiment, the compounds disclosed herein may be prepared following Schemes 1-4, using suitable starting materials known in the art and/or available from a commercial source. In one embodiment, the compounds disclosed herein may be prepared from suitable starting materials and following Schemes 5-7.

The following values are used for the substituents drawn in the Schemes:
n1=1 or 2; R⁶=CH₃; w=0, 1, or 2; p=0; R₉=H, CH₃, CH₂CH₃; R¹⁰=H, CH₃, CH₂CH₃; Ring B=a 6-membered aromatic ring, unsubstituted or substituted with halo; or an unsubstituted 5- or 6-membered heteroaromatic ring having 1 to 3 ring heteroatoms independently selected from O, N and S.

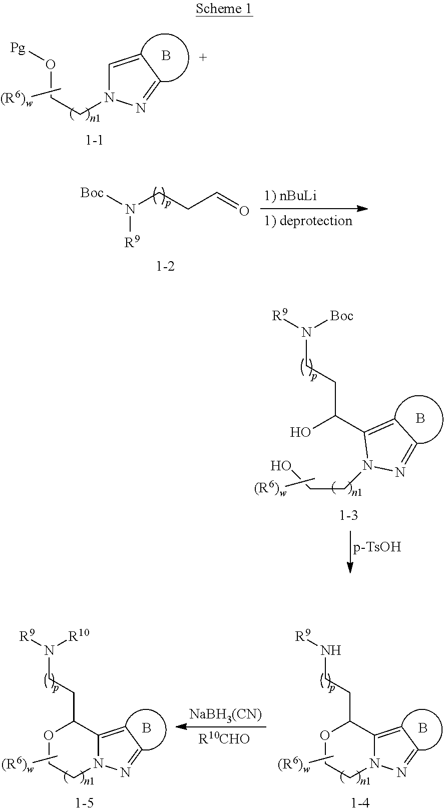

In one embodiment, a suitable O-protected hydroxyalkyl substituted pyrazole (1-1) is treated with a lithium reagent such as n-BuLi, followed by reaction with an aminoacetaldehyde or N-protected aminoacetaldehyde (1-2) and then removal of the O-protecting group to give compound 1-3 (Scheme 1). Treatment of 1-3 with p-TsOH provides a cyclized compound (compound 1-4). Compound 1-4 is separated using chiral HPLC or SFC chromatography to provide single enantiomers. In another embodiment, 1-4 is N-protected with a BOC-protecting group by reacting 1-4 with di-tent-butyl dicarbonate. After chemical purification or chiral separation, the BOC-group is removed under acidic conditions to afford compound 1-4, in the case of chemical purification, or the single enantiomers of compound 1-4, in the case of chiral separation. In other embodiments, compound 1-4 or single enantiomers derived from 1-4 are N-alkylated such as with sodium cyanoborohydride and an aldehyde such as formaldehyde, acetaldehyde or propionaldehyde to provide compound 1-5 or single enantiomers of compound 1-5.

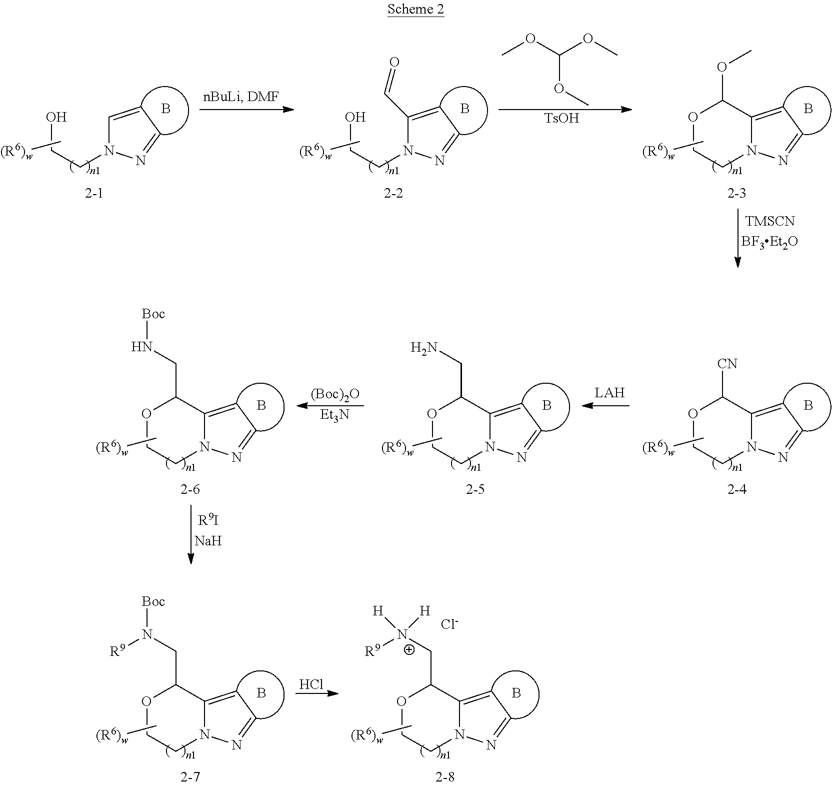

In one embodiment, a suitable hydroxyalkyl substituted pyrazole (2-1) is treated with a lithium reagent such as n-BuLi and DMF to give aldehyde 2-2. Reaction of 2-2 with trimethoxymethane and p-toluenesulfonic acid provides methyl ether 2-3. Compound 2-3 reacted with trimethylsilylcyanide and boron trifluoride etherate provides compound 2-4. Compound 2-4 is reduced to an amine compound (compound 2-5). Compound 2-5 is separated into single enantiomers such as described in Scheme I. In another embodiment, compound 2-5 is further functionalized such as by N-Boc protection (to compound 2-6), alkylation (to compound 2-7) and deprotection to compound 2-8. In another embodiment, compound 2-8 is separated into single enantiomers such as described in Scheme 1 to provide compounds disclosed herein that are single enantiomers.

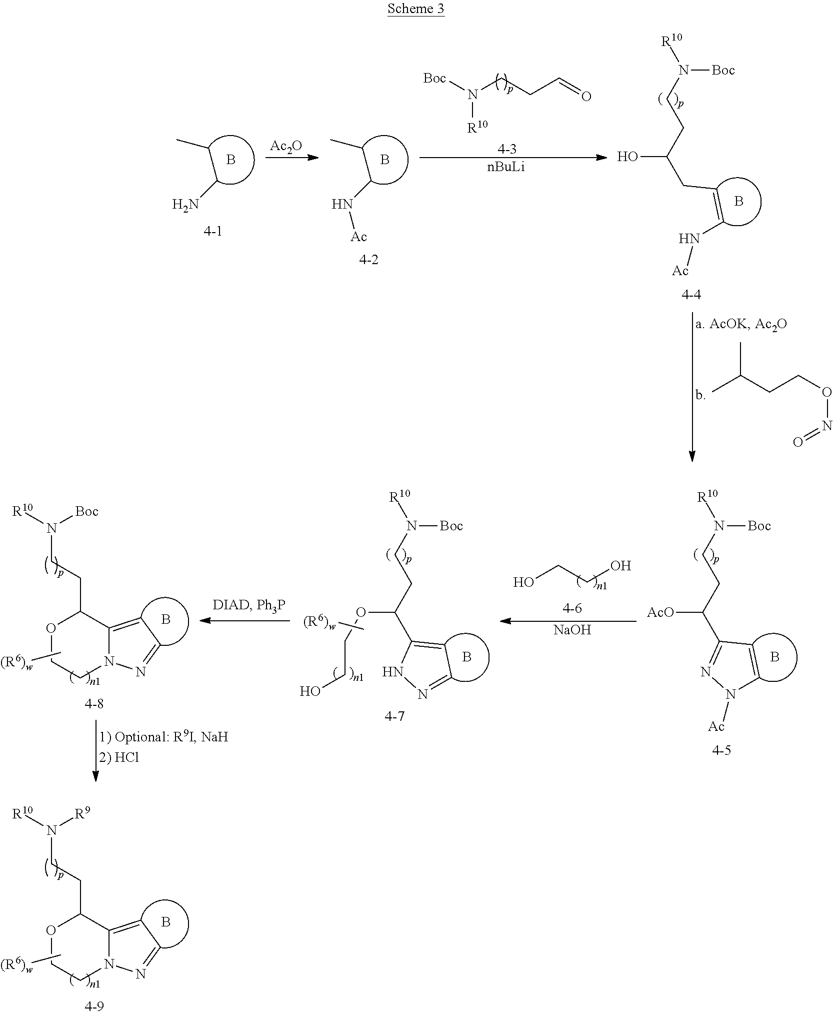

In one embodiment, the amine of a suitable α-aminomethyl-substituted aromatic or heteroaromatic compound (4-1) is acylated in acetic anhydride to form compound 4-2. Compound 4-2 is treated with a lithium reagent such as n-BuLi and amino-protected aldehyde 4-3 to provide alcohol 4-4, which is then converted to the corresponding fused pyrazole 4-5 with isoamyl nitrite. Substitution with diol 4-6 provides 4-7, which is then subjected to Mitsunobu conditions with diisopropyl azodicarboxylate and triphenylphosphine to trigger cyclization to compound 4-8. Compound 4-8 is then alkylated and then deprotected as in Scheme 2 to form compound 4-9. In another embodiment, compound 4-8 is deprotected as in Scheme 2 to form compound 4-9, where R⁹=H. In another embodiment, compound 4-9 is separated into single enantiomers disclosed herein such as described in Scheme 1.

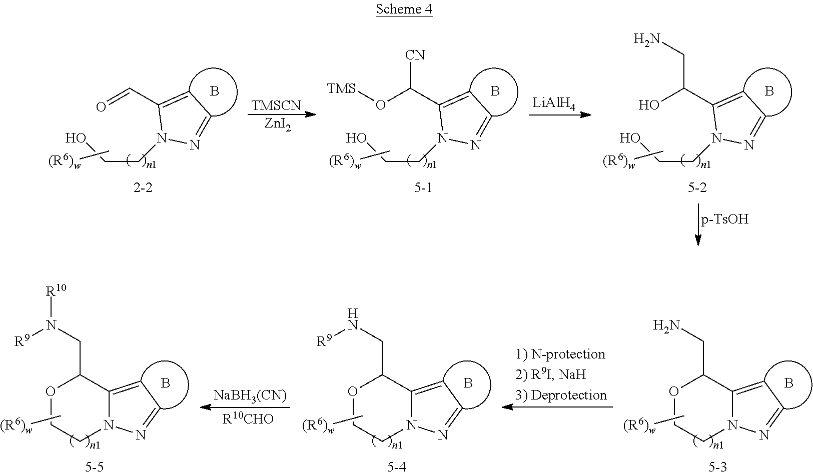

In one embodiment, aldehyde 2-2 from Scheme 2 is treated with TMSCN with ZnI₂ to provide 5-1. The cyano group of compound 5-1 is then reduced with lithium aluminum hydride to provide compound 5-2. Cyclization of 5-2 with p-TsOH provides compound 5-3. In another embodiment, compound 5-3 is separated into single enantiomers such as described in Scheme 1. In another embodiment, the amine of compound 5-3 or the separated enantiomers of compound 5-3 are protected as in Scheme 2, alkylated by treatment with sodium hydride and an alkyl halide as in Scheme 2, then deprotected as in Scheme 2 to provide 5-4. In another embodiment, a racemic mixture 5-4 is separated into single enantiomers disclosed herein such as described in Scheme 1. In another embodiment, compound 5-4 or the separated enantiomers of compound 5-4 are N-alkylated as in Scheme 1 to form compound 5-5. In another embodiment, a racemic mixture 5-5 is separated into single enantiomers disclosed herein as described in Scheme 1.

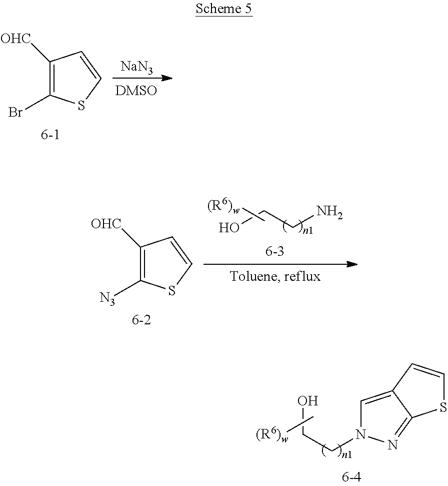

In one embodiment, a compound disclosed herein is prepared from thienopyrazole compound 6-4. In one embodiment, compound 6-4 is synthesized in two steps from bromothiophene-3-carbaldehyde 6-1. Compound 6-1 is converted to compound 6-2 with sodium azide, which is then converted to compound 6-3 by refluxing with amino alcohol 6-3. In one embodiment, n1=1 and R⁶=H.

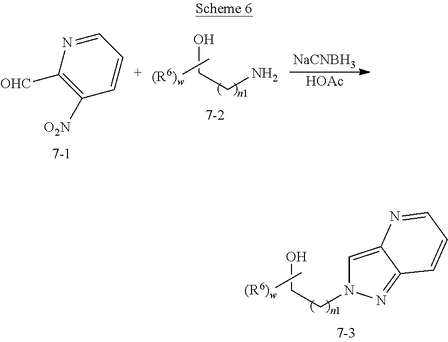

In one embodiment, the compound disclosed herein is prepared from pyrazolopyridine compound 7-3. In one embodiment, 3-nitropicolinaldehyde 7-1 and amino alcohol 7-2 are reacted with sodium cyanoborohydride and acetic acid to form compound 7-3. In one embodiment, n1=1 and R⁶=H.

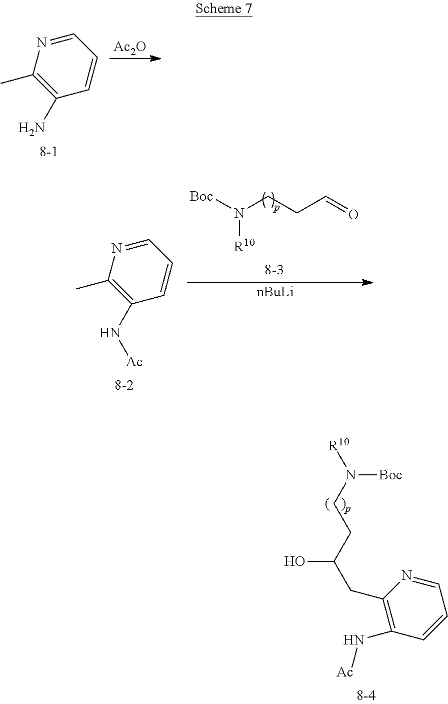

In one embodiment, the compound disclosed herein is prepared from pyridyl compound 8-4. In one embodiment, 2-methylpyridin-3-amine (8-1) is acylated with acetic anhydride to produce 8-2. Compound 8-2 is treated with a lithium reagent such as n-BuLi and aldehyde 8-3 to provide pyridyl compound 8-4. In one embodiment, R¹⁰=CH₃ and p=0.

In some embodiments, compounds are prepared according to these procedures. It will be appreciated that, although the general methods depict the synthesis of certain compounds of the present invention, these methods, and other methods known to persons skilled in the art, can be applied to all compounds.

### Example 2. Neuropharmacological Assay (SmartCube™)

One having ordinary skill in the art would recognize that there is a plurality of ways to test a compound's efficacy in treating neurological and psychiatric diseases and disorders. The following non-limiting example provides study designs to measure efficacy of the compound disclosed herein for treating neurological and psychiatric diseases and disorders. The study design is incorporated by reference in its entirety.

Exemplary compounds are evaluated using the neuropharmacological screen described in S. L. Roberds et al., Front. Neurosci. 2011 Sep. 9; 5:103 (doi: 10.3389/fnins.2011.00103) (“Roberds”). As reported in Roberds, because psychiatric diseases generally result from disorders of cell-cell communication or circuitry, intact systems are useful in detecting improvement in disease-relevant endpoints. These endpoints are typically behavioral in nature, often requiring human observation and interpretation. To facilitate testing of multiple compounds for behavioral effects relevant to psychiatric disease, PsychoGenics, Inc. (Tarrytown, NY, “PGI”) developed SmartCube™, an automated system in which behaviors of compound-treated mice are captured by digital video and analyzed with computer algorithms. (D. Brunner et al., Drug Discov. Today 2002, 7:S107-S112). PGI Analytical Systems uses data from SmartCube™ to compare the behavioral signature of a test compound to a database of behavioral signatures obtained using a large set of diverse reference compounds. (The composition of the database as well as validation of the method is further described in Roberds). In this way, the neuropharmacological effects of a test compound can be predicted by similarity to major classes of compounds, such as antipsychotics, anxiolytics and antidepressants.

The SmartCube™ system produces an activity signature indicating the probability that the activity of the test compound at the administered dose matches a given class of neuropharmacological agents. (See, e.g., Roberds, FIGS. 2 and 3). The test compound is simultaneously compared against multiple classes of agents; thus, a separate probability is generated for each behavioral effect measured (e.g., anxiolytic activity, analgesic activity, etc.). Probabilities are reported for each behavioral effect measured. Provided compounds are dissolved in a mixture of Pharmasolve™ (N-methyl-2-pyrrolidone), polyethylene glycol and propylene glycol, and are injected i.p. 15 min. to mice before the behavioral test. For each compound, injections are administered at 3 different doses. It is expected that at least some of the compounds of the present disclosure would have one or more properties such as DP: anti-depressant; AX: anxiolytic; SD: sedative hypnotic; PS: anti-psychotic; MS: mood stabilizer; AD: ADHD; CE: cognitive enhancer; AG: analgesic; and/or UN: uncharacterized CNS activity.

In some embodiments, the recitation of a “compound” is intended to encompass both free compounds and pharmaceutically acceptable salts thereof. As a convention, the phrase “or a pharmaceutically acceptable salt thereof” is explicitly recited when the structural formula of the compound is explicitly recited, but no difference in inclusion or exclusion of pharmaceutically acceptable salts is thereby intended.

It may be found upon examination that additional species and genera not presently excluded from the claims to pharmaceutical compositions and chemical compounds are not patentable to the inventors in this application. In that case, the subsequent exclusion of species and genera in applicants' claims are to be considered artifacts of patent prosecution and not reflective of the inventors' concept or description of their invention. The invention, in a composition aspect, is all compounds disclosed herein (e.g., of Table 1) except those that are in the public's possession.

It is to be understood that the invention is not limited to the particular embodiments of the invention described above, as variations of the particular embodiments may be made and still fall within the scope of the appended claims.

What is claimed is:

1. A compound of Table 1, wherein said compound is of structure:
TABLE 1

| | |
|---|---|
| 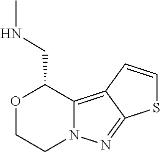 | 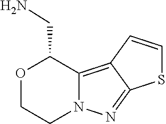 [SMILES: NC[C@H]1OCCn2nc3sccc3c21] |
| 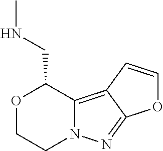 | 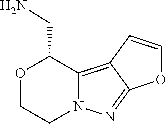 [SMILES: NC[C@H]1OCCn2nc3occc3c21] |
| 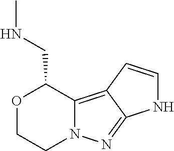 | 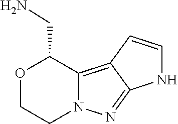 [SMILES: NC[C@H]1OCCn2nc3[nH]ccc3c21] |
| 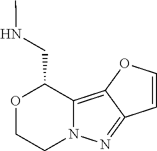 | 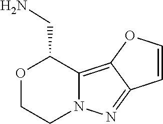 [SMILES: NC[C@H]1OCCn2nc3ccoc3c21] |
| 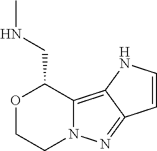 | 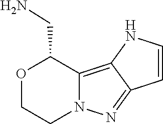 [SMILES: NC[C@H]1OCCn2nc3cc[nH]c3c21] |
| 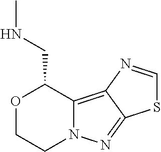 | 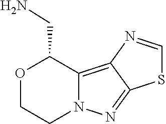 [SMILES: NC[C@H]1OCCn2nc3scnc3c21] |
| 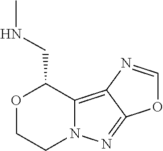 | 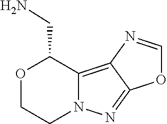 [SMILES: NC[C@H]1OCCn2nc3ocnc3c21] |
| 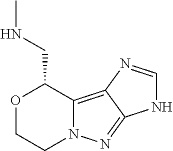 | 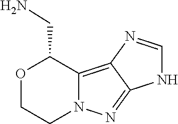 [SMILES: NC[C@H]1OCCn2nc3[nH]cnc3c21] |
| 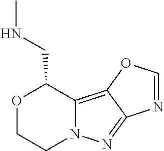 | 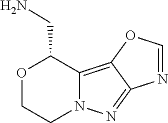 [SMILES: NC[C@H]1OCCn2nc3ncoc3c21] |
| 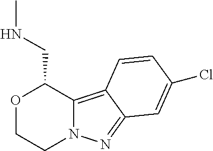 [SMILES: CNC[C@H]1OCCn2nc3cc(Cl)ccc3c21] | 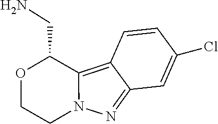 [SMILES: NC[C@H]1OCCn2nc3cc(Cl)ccc3c21] |
| 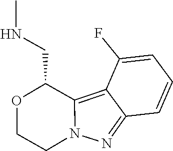 [SMILES: CNC[C@H]1OCCn2nc3cccc(F)c3c21] | 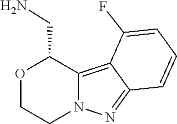 [SMILES: NC[C@H]1OCCn2nc3cccc(F)c3c21] |
| 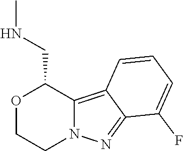 [SMILES: CNC[C@H]1OCCn2nc3c(F)cccc3c21] | 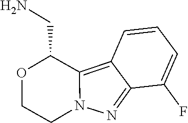 [SMILES: NC[C@H]1OCCn2nc3c(F)cccc3c21] |
| 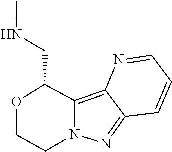 [SMILES: CNC[C@H]1OCCn2nc3cccnc3c21] | 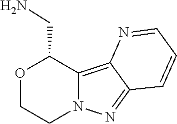 [SMILES: NC[C@H]1OCCn2nc3cccnc3c21] |
| 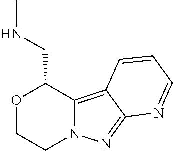 [SMILES: CNC[C@H]1OCCn2nc3ncccc3c21] | 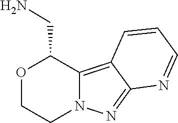 [SMILES: NC[C@H]1OCCn2nc3ncccc3c21] |
| 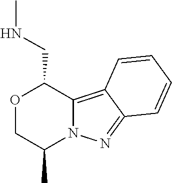 [SMILES: CNC[C@H]1OC[C@H](C)n2nc3ccccc3c21] | 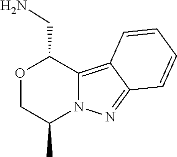 [SMILES: C[C@H]1CO[C@H](CN)c2c3ccccc3nn21] |
| 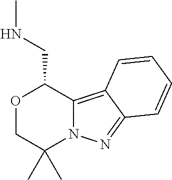 [SMILES: CNC[C@H]1OCC(C)(C)n2nc3ccccc3c21] | 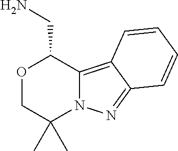 [SMILES: CC1(C)CO[C@H](CN)c2c3ccccc3nn21] |
| 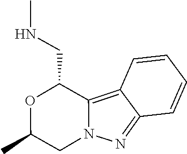 [SMILES: CNC[C@H]1O[C@H](C)Cn2nc3ccccc3c21] | 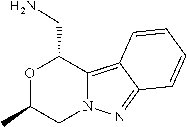 [SMILES: C[C@@H]1Cn2nc3ccccc3c2[C@@H](CN)O1] |
| 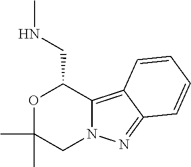 [SMILES: CNC[C@H]1OC(C)(C)Cn2nc3ccccc3c21] | 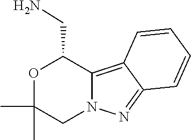 [SMILES: CC1(C)Cn2nc3ccccc3c2[C@@H](CN)O1] |
| 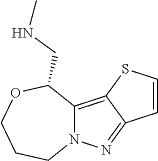 [SMILES: CNC[C@H]1OCCCn2nc3ccsc3c21] | 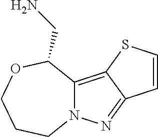 [SMILES: NC[C@H]1OCCCn2nc3ccsc3c21] |
| 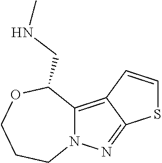 [SMILES: CNC[C@H]1OCCCn2nc3sccc3c21] | 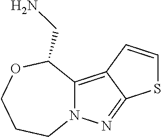 [SMILES: NC[C@H]1OCCCn2nc3sccc3c21] |
| 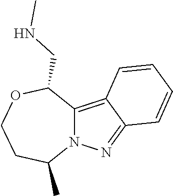 [SMILES: CNC[C@H]1OCC[C@H](C)n2nc3ccccc3c21] | 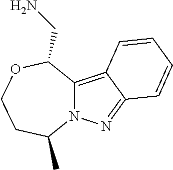 [SMILES: C[C@H]1CCO[C@H](CN)c2c3ccccc3nn21] |
| 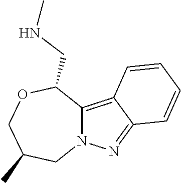 [SMILES: CNC[C@H]1OC[C@H](C)Cn2nc3ccccc3c21] | 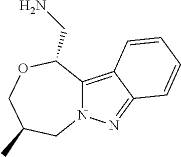 [SMILES: C[C@H]1CO[C@H](CN)c2c3ccccc3nn2C1] |
| 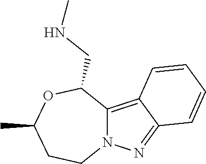 [SMILES: CNC[C@H]1O[C@H](C)CCn2nc3ccccc3c21] | 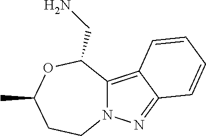 [SMILES: C[C@@H]1CCn2nc3ccccc3c2[C@@H](CN)O1] |
| 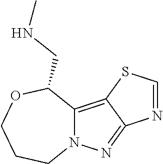 [SMILES: CNC[C@H]1OCCCn2nc3ncsc3c21] | 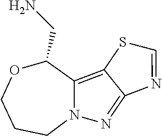 [SMILES: NC[C@H]1OCCCn2nc3ncsc3c21] |
| 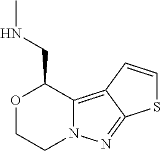 | 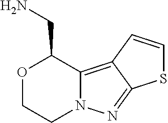 [SMILES: NC[C@@H]1OCCn2nc3sccc3c21] |
| 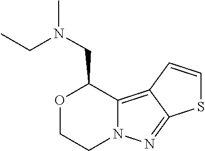 [SMILES: CCN(C)C[C@@H]1OCCn2nc3sccc3c21] | 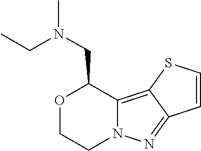 [SMILES: CCN(C)C[C@@H]1OCCn2nc3ccsc3c21] |
| 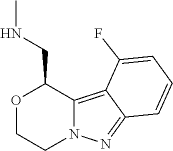 [SMILES: CNC[C@@H]1OCCn2nc3cccc(F)c3c21] | 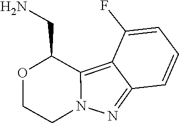 [SMILES: NC[C@@H]1OCCn2nc3cccc(F)c3c21] |
| 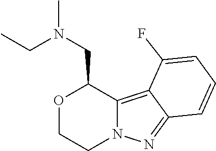 [SMILES: CCN(C)C[C@@H]1OCCn2nc3cccc(F)c3c21] | 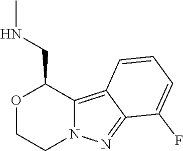 [SMILES: CNC[C@@H]1OCCn2nc3c(F)cccc3c21] |
| 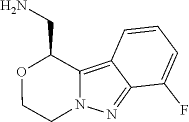 [SMILES: NC[C@@H]1OCCn2nc3c(F)cccc3c21] | 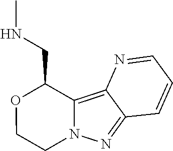 [SMILES: CNC[C@@H]1OCCn2nc3cccnc3c21] |
| 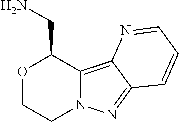 [SMILES: NC[C@@H]1OCCn2nc3cccnc3c21] | 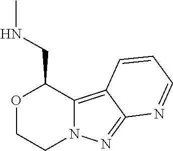 [SMILES: CNC[C@@H]1OCCn2nc3ncccc3c21] |
| 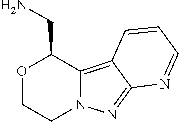 [SMILES: NC[C@@H]1OCCn2nc3ncccc3c21] | 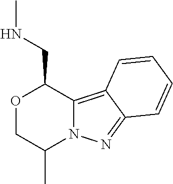 [SMILES: CNC[C@@H]1OCC(C)n2nc3ccccc3c21] |
| 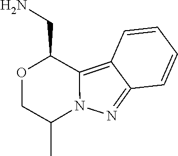 [SMILES: CC1CO[C@@H](CN)c2c3ccccc3nn21] | 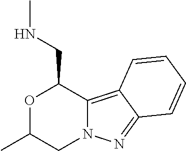 [SMILES: CNC[C@@H]1OC(C)Cn2nc3ccccc3c21] |
| 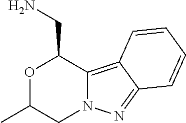 [SMILES: CC1Cn2nc3ccccc3c2[C@H](CN)O1] | 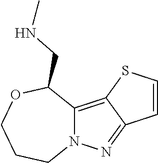 [SMILES: CNC[C@@H]1OCCCn2nc3ccsc3c21] |
| 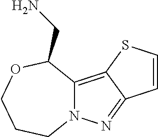 [SMILES: NC[C@@H]1OCCCn2nc3ccsc3c21] | 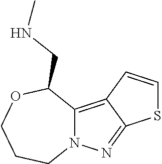 [SMILES: CNC[C@@H]1OCCCn2nc3sccc3c21] |
| or | 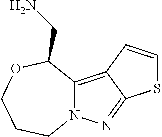 [SMILES: NC[C@@H]1OCCCn2nc3sccc3c21] |

or a pharmaceutically acceptable salt thereof.

2. The compound according to claim 1, wherein said compound is selected from: 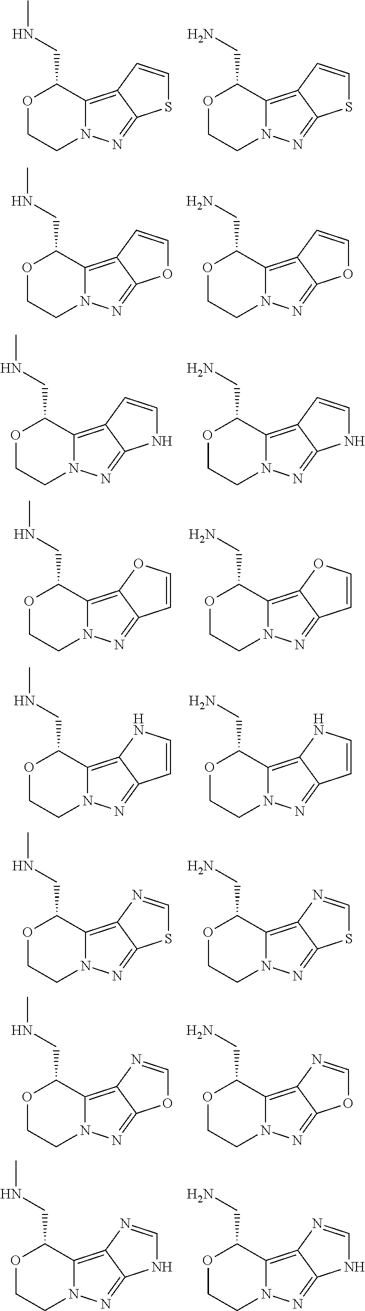 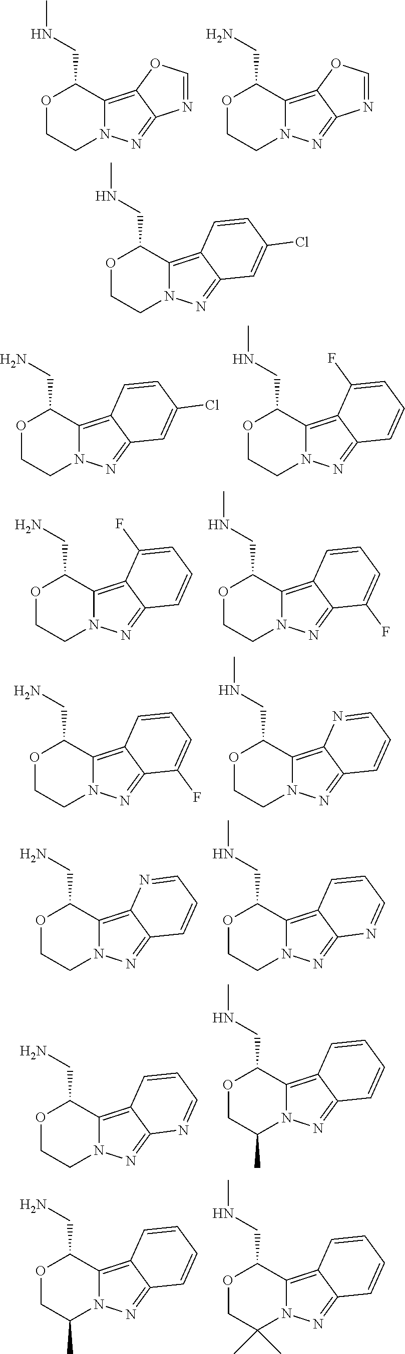 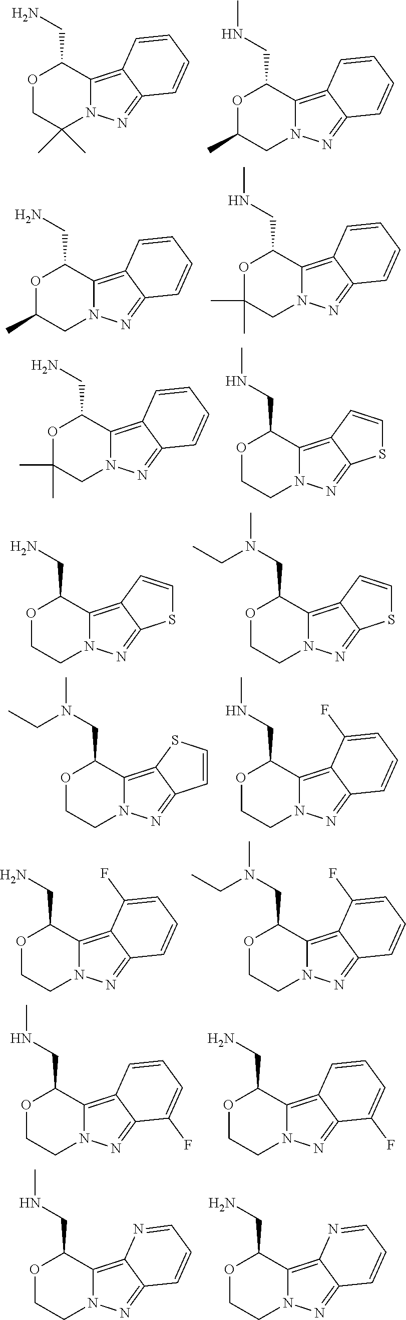 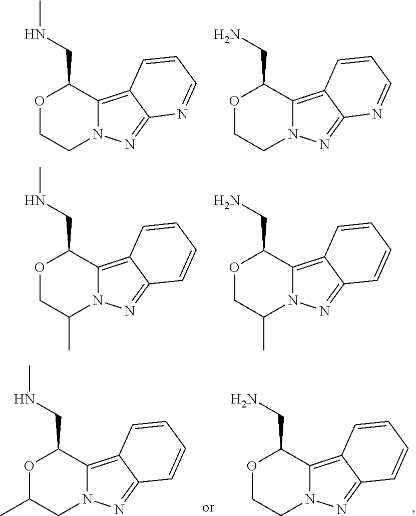
or a pharmaceutically acceptable salt thereof.

3. The compound according to claim 1, wherein said compound is selected from: 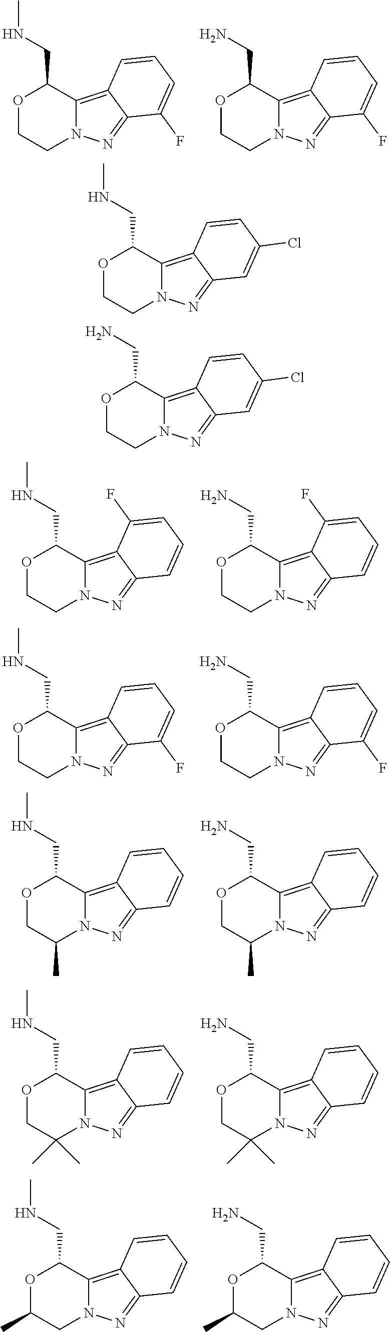 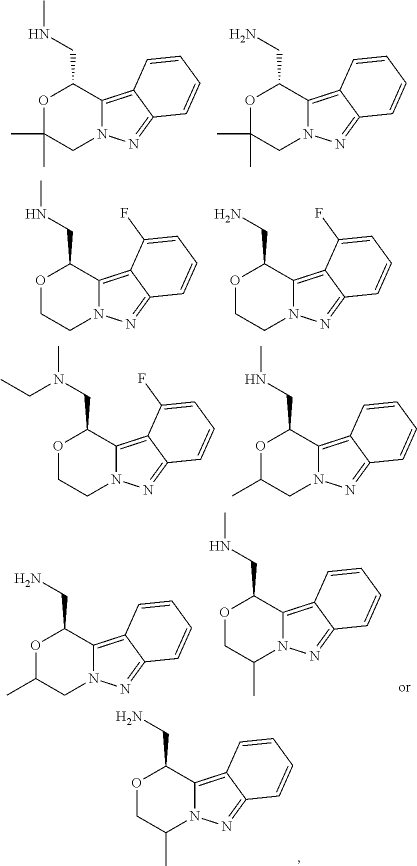
or a pharmaceutically acceptable salt thereof.

4. The compound according to claim 1, wherein said compound is selected from: 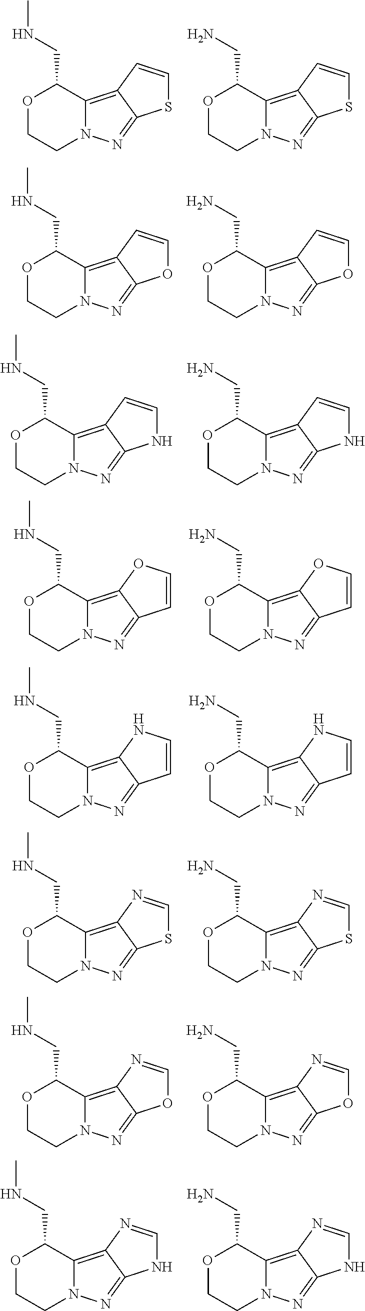 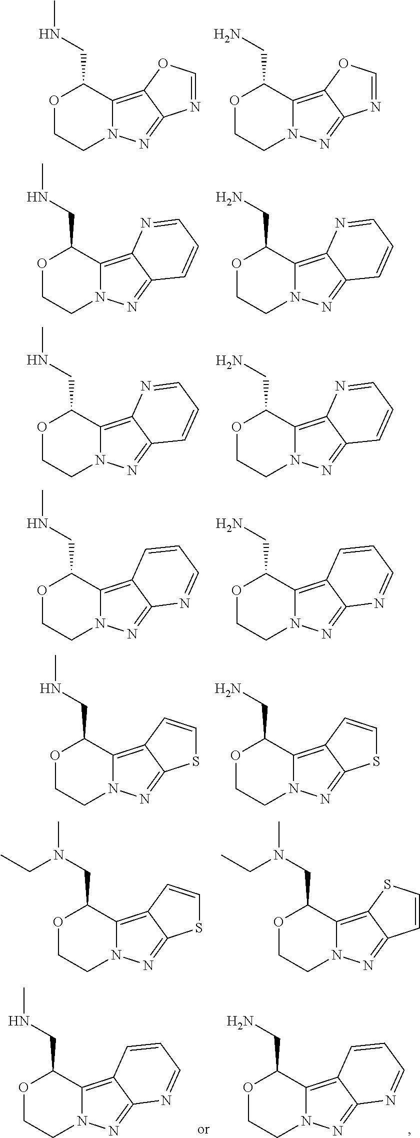
or a pharmaceutically acceptable salt thereof.

5. The compound according to claim 1, wherein said compound is selected from: 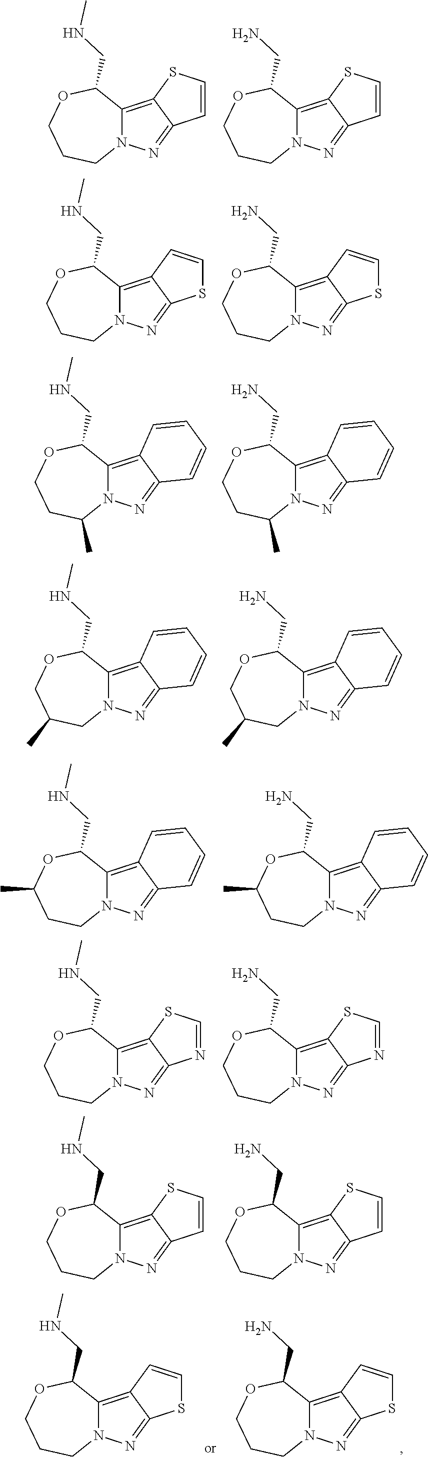
or a pharmaceutically acceptable salt thereof.

6. The compound according to claim 1, wherein said compound is selected from: 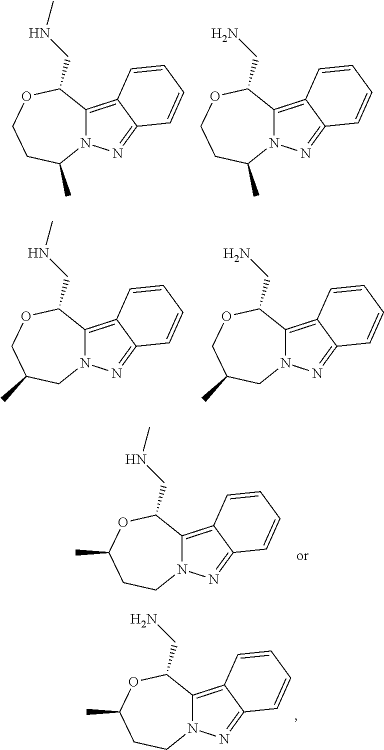
or a pharmaceutically acceptable salt thereof.

7. The compound according to claim 1, wherein said compound is selected from: 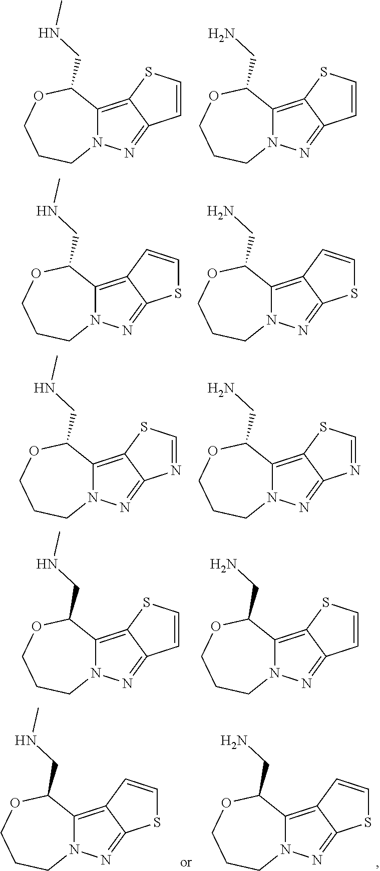
or a pharmaceutically acceptable salt thereof.

8. A composition comprising the compound of claim 1, or a pharmaceutically acceptable salt thereof, and a pharmaceutically acceptable carrier, adjuvant, or vehicle.

9. A method for treating a neurological or psychiatric disorder in a patient, comprising administering to said patient an effective amount of one or more compounds of claim 1, or a pharmaceutically acceptable salt thereof.

10. A method for treating a neurological or psychiatric disorder in a patient, comprising administering to said patient an effective amount of a composition of claim 8.

11. A method for treating a neurological or psychiatric disorder in a patient, comprising administering to said patient an effective amount of one or more compounds of claim 2, or a pharmaceutically acceptable salt thereof.

12. A method for treating a neurological or psychiatric disorder in a patient, comprising administering to said patient an effective amount of one or more compounds of claim 3, or a pharmaceutically acceptable salt thereof.

13. A method for treating a neurological or psychiatric disorder in a patient, comprising administering to said patient an effective amount of one or more compounds of claim 4, or a pharmaceutically acceptable salt thereof.

14. A method for treating a neurological or psychiatric disorder in a patient, comprising administering to said patient an effective amount of one or more compounds of claim 5, or a pharmaceutically acceptable salt thereof.

15. A method for treating a neurological or psychiatric disorder in a patient, comprising administering to said patient an effective amount of one or more compounds of claim 6, or a pharmaceutically acceptable salt thereof.

16. A method for treating a neurological or psychiatric disorder in a patient, comprising administering to said patient an effective amount of one or more compounds of claim 7, or a pharmaceutically acceptable salt thereof.
